(19) United States
(12) Patent Application Publication
(10) Pub. No.: US 2016/0198739 A1
(43) Pub. Date: Jul. 14, 2016

(54) METHODS FOR FEEDING DIETARY COMPOSITIONS TO RUMINANTS

(71) Applicant: BENEMILK OY, Raisio (FI)

(72) Inventors: Feng Wan, Issaquah, WA (US); Ilmo Pellervo Aronen, Hinnerjoki (FI); Merja Birgitta Holma, Raisio (FI); Christopher John Buntel, Singapore (SG)

(73) Assignee: Benemilk Oy, Raisio (FI)

(21) Appl. No.: 14/908,102

(22) PCT Filed: Jul. 30, 2013
(86) PCT No.: PCT/US2013/052680

§ 371 Date: Jan. 27, 2016

(51) Int. Cl. A23K 20/158; A01K 29/00; A01K 11/00; A23K 50/10; A01K 5/02

(52) U.S. Cl. CPC A23K 20/158; A23K 50/10; A01K 5/0291; A01K 11/006; A01K 29/005

(57) ABSTRACT

Dietary compositions for ingestion by ruminants and methods for feeding the dietary compositions to ruminants are disclosed. The dietary compositions may include ruminant feed products having at least one saturated fatty acid component including at least about 90% of a palmitic acid compound. The ruminant feed product may be fed to the ruminant according to a feed program that is based on physical characteristics and/or a time period associated with the ruminant. For example, an amount of the ruminant feed product fed to the ruminant may be based on the calving cycle and/or lactation cycle of the ruminant and one of a body weight or a milk yield of the ruminant. Ingestion of the ruminant feed product by a ruminant according to the feed program may increase the milk yield and/or the milk fat content of milk produced by the ruminant.

### BACKGROUND

Increasing production and fat content of milk obtained from lactating ruminants has been a major goal for dairy farmers. Additional milk production per ruminant is beneficial because it results in a higher yield, thereby increasing profits. Increased milk fat is desirable because it has a higher economic value and can be used in highly desirable food products, such as cheese, yogurt, and the like.

Conventional approaches to increasing production yield and milk fat content includes adjusting feed, nutrients, elements, vitamins, supplements, and/or the like provided to the ruminant. However, the current methods and feeds used to increase milk fat content tend to lower milk production or vice versa, and do not adequately take the physical conditions and time periods associated with the ruminants into account. In addition, these methods and feeds may produce other detrimental effects, such as lower protein content, taste defects, preservation issues, and increased trans fatty acid levels. Accordingly, the milk production industry would benefit from a method of feeding ruminants that increases milk yield and milk fat content without producing negative consequences that may outweigh the benefits of the feed.

### SUMMARY

In an embodiment, a method for feeding a ruminant may include determining a body weight of the ruminant and a milk yield of the ruminant, and providing a ruminant feed product to the ruminant for ingestion according to a feed program. The ruminant feed product may comprise at least one fatty acid component comprising at least about 70% of a palmitic acid compound by weight. The feed program may comprise feeding the ruminant feed product to the ruminant at a first level during a first time period, wherein the first level does not exceed about 0.04 grams of the fatty acid component per kilogram of the body weight, feeding the ruminant feed product to the ruminant at a second level during a second time period, wherein the second level comprises at least 0.04 grams of the fatty acid component per kilogram of the body weight, and feeding the ruminant feed product to the ruminant at a third level during a third time period, wherein the third level comprises at least 0.4 grams of the fatty acid component per kilogram of the body weight.

In an embodiment, a method of feeding a ruminant may include determining a body weight of the ruminant and a milk yield of the ruminant, and providing a ruminant feed product to the ruminant for ingestion according to a feed program. The feed product may comprise at least one fatty acid component comprising at least about 70% of a palmitic acid compound by weight. The feed program may comprise feeding the ruminant feed product to the ruminant at a first level of at most about 0.04 grams of the fatty acid component per kilogram of the body weight during a first time period, wherein the first time period comprises a far-off dry cow period, feeding the ruminant feed product to the ruminant at a second level of at least 0.04 grams of the fatty acid component per kilogram of the body weight during a second time period, wherein the second time period comprises a close-up dry cow period, and feeding the ruminant feed product to the ruminant at a third level of at least 0.4 grams of fatty acid component per kilogram of the body weight during a third time period, wherein the third time period comprises at least one of a fresh cow period, a peak lactation period, a constant phase of lactation period, and a late lactation period.

In an embodiment, a system for feeding a ruminant may comprise a feed source configured to provide a ruminant feed product for ingestion by the ruminant, the ruminant feed product comprising at least one fatty acid component comprising at least about 70% of a palmitic acid compound by weight, and at least one feeding element having access to the feed source, the at least one feeding element being configured to feed the ruminant feed product to the ruminant according to a feed program. The feed program may comprise feeding the ruminant feed product to the ruminant at a first level during a first time period, wherein the first level does not exceed about 0.04 grams of the fatty acid component per kilogram of the body weight, feeding the ruminant feed product to the ruminant at a second level during a second time period, wherein the second level comprises at least 0.04 grams of the fatty acid component per kilogram of the body weight, and feeding the ruminant feed product to the ruminant at a third level during a third time period, wherein the third level comprises at least 0.4 grams of the fatty acid component per kilogram of the body weight.

### BRIEF DESCRIPTION OF THE DRAWINGS

FIG. 1 depicts a flow diagram of an illustrative method of feeding a ruminant according to a first embodiment.

FIG. 2 depicts a flow diagram of an illustrative method of feeding a ruminant according to a second embodiment.

FIG. 3 depicts a flow diagram of illustrative feed program according to a first embodiment.

FIG. 4 depicts a flow diagram of illustrative feed program according to a second embodiment.

FIG. 5 depicts an illustrative lactation cycle for a ruminant and a feed program for feeding the ruminant according to some embodiments.

### DETAILED DESCRIPTION

This disclosure is not limited to the particular systems, devices and methods described, as these may vary. The terminology used in the description is for the purpose of describing the particular versions or embodiments only, and is not intended to limit the scope.

As used in this document, the singular forms “a,” “an,” and “the” include plural references unless the context clearly dictates otherwise. Unless defined otherwise, all technical and scientific terms used herein have the same meanings as commonly understood by one of ordinary skill in the art. Nothing in this disclosure is to be construed as an admission that the embodiments described in this disclosure are not entitled to antedate such disclosure by virtue of prior invention. As used in this document, the term “comprising” means “including, but not limited to.”

The following terms shall have, for the purposes of this application, the respective meanings set forth below.

A “ruminant” is generally a suborder of mammal with a multiple chamber stomach that gives the animal the ability to digest cellulose-based food by softening it within a first chamber (rumen) of the stomach and to regurgitate the semi-digested mass to be chewed again by the ruminant for digestion in one or more other chambers of the stomach. Examples of ruminants include, but are not limited to, lactating animals such as cattle, goats and sheep. Cattle may include dairy cows, which are generally animals of the species Bos taurus. The milk produced by ruminants is widely used in a variety of dairy-based products.

The present technology generally relates to methods for feeding dietary compositions to ruminants. The methods for feeding the dietary compositions may be configured to improve various aspects of milk production in the ruminants. For instance, some embodiments provide that the methods of feeding the dietary compositions may increase the amount of milk produced by the ruminant (for example, the milk yield) and/or increase the fat content of the milk produced by the ruminant. The dietary compositions may include ruminant feed product having a saturated fatty acid component that include at least about 70% of a palmitic acid compound by weight. The ruminant feed product may be configured to improve various aspects of milk production in the ruminants and may more considerably improve various aspects of milk production in the ruminants when fed according to feeding methods described herein.

The diet ingested by a ruminant may have different effects on the ruminant during different phases of its lifecycle and lactation cycle. Accordingly, methods for feeding ruminants provided according to some embodiments described herein may be configured to feed the ruminant feed product to the ruminant at various concentrations based on, among other things, various time periods associated with the ruminant. In an embodiment, the time periods may be based on the calving cycle of the ruminant. For instance, the ruminant may be fed certain concentrations of the ruminant feed product at different time periods before calving or different concentrations at different time periods after calving. However, embodiments are not limited to time periods based on the calving cycle as any time period capable of operating according to some embodiments is contemplated herein. For instance, time periods may include, without limitation, seasonal time periods, life stages (for example, juvenile, adult, or the like), health-based time periods (for example, the ruminant may be fed different concentrations of the ruminant feed product during times of good health as compared to times of illness), or the like. The concentrations may be determined based on, for example, the weight of the ruminant and/or the milk yield of the ruminant. In an embodiment, the milk yield may include kilograms of milk produced per day, per week, per year, or the like. For example, at a certain time period the ruminant may be fed an amount of ruminant feed product (for instance, grams) for every unit of weight of the ruminant (for example, kilograms) or every unit of milk production (for example, kilograms/day). In this manner, the milk yield and/or the fat content of the milk produced by the ruminant may be maximized based on certain time periods and characteristics (for example, body weight and milk yield) associated with the ruminant without encountering various deleterious effects associated with conventional feed and feeding methods.

The physical and/or dietary needs of a ruminant, such as a dairy cow, change as the cow progresses through the calving cycle, gives birth (calves), and enters the post-calving period. Increasing the fat content of the milk right after calving may cause a negative energy balance for the cow if not managed with proper feeding. The feed and feeding methods may have an effect on the quality of colostrum (for example, milk produced by a cow for a newborn calf) produced by the ruminant after calving. Colostrum is crucial for newborn calves because they do not experience the passive transfer of immunity via the placenta before birth, so any antibodies that they need have to be ingested. Increased amounts of high quality colostrum may decrease calf mortality and may allow the calves to grow faster and healthier. In addition, the high fat colostrum is a very important energy source for the calf because the body reserves of the calf are very limited. Accordingly, methods for feeding a ruminant may be configured to maximize fat content and milk yield while based on, among other things, the calving cycle of the ruminant.

When a ruminant consumes feed, the fat in the feed is modified by the rumen to provide a milk fat profile that is different from the profile of fat in the feed. All fats which are not completely inert in the rumen may decrease feed intake and rumen digestibility of the feed material. Milk composition and fat quality may be influenced by the ruminant's diet. For example, oil feeding (for instance, the feeding of vegetable oils) can have negative effects on both rumen function and milk formation. As a result of oil feeding, the milk protein concentration may be lowered, the fat concentration may be decreased, and the proportion of trans fatty acids may be increased. These results have been connected with various negative milk characteristics, such as an increase in the harmful low-density lipoprotein (LDL) cholesterol and a decrease in the beneficial high-density lipoprotein (HDL) cholesterol in human blood when the milk is consumed. In addition, the properties of the milk fat during industrial milk processing may be weakened. A high level of polyunsaturated fatty acids in milk can also cause taste defects and preservation problems. A typical fatty acid composition of milk fat may contain more than about 70% saturated fatty acids and a total amount of trans fatty acids may be from about 3% to about 10%. When vegetable oil is added into the feed, the proportion of trans fatty acids may rise to more than about 10%.

One solution to diminishing the detrimental effect of oil and fat is to prevent triglyceride fat hydrolysis. Fat hydrolysis can be decreased, for example, by protecting fats with formaldehyde treated casein. Another alternative is to feed the ruminant insoluble fatty acid calcium salts whereby hydrogenation in rumen can be avoided. However, fatty acid salts typically have a pungent taste that may result in decreased feed intake by the ruminant.

Accordingly, the ruminant feed product described herein may include a saturated fatty acid component that includes at least about 70% to about 100% of a palmitic acid compound by weight. In an embodiment, the saturated fatty acid component may include at least about 90% of the palmitic acid compound by weight. The dietary composition allows for the transfer of palmitic acid from the feed via the digestive tract into the blood circulation of a ruminant. This may improve the energy efficiency of milk production and the utilization of energy of the ruminant. When the utilization of energy becomes more effective, milk production may increase and/or the concentrations of protein and/or fat in the milk may rise. According to some embodiments, the dietary composition may be configured to enhance fat synthesis in the mammary gland by bringing milk fat components to the cell such that energy consuming synthesis in the mammary gland is not necessary. As a result, glucose may be used more efficiently for lactose production causing increased milk production. In addition, the milk protein content may increase because there is no need to produce glucose from amino acids. Thus, the ruminant therefore does not lose weight or loses less weight at the beginning of the lactation period, thereby improving the fertility of the ruminant.

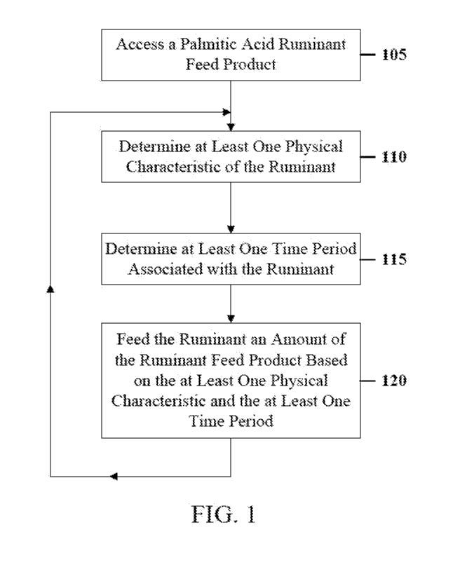

FIG. 1 depicts a flow diagram of an illustrative method for feeding a ruminant according to a first embodiment. In various embodiments, the components described herein with respect to FIG. 1 may generally be combined in any order and/or any combination, may include more or fewer components, and are not limited by the order described herein. In various embodiments, the feeding methods may be configured in a manner that maximizes particular qualities in the milk produced by the ruminant, as well as an amount of milk produced by the ruminant, as described in greater detail herein.

As depicted in FIG. 1, a palmitic acid ruminant feed product (the “ruminant feed product”) may be accessed 105 for feeding to the ruminant The ruminant feed product may be accessed 105 in various forms, such as a fluid, a dry or substantially dry material, pellets, loose particulates, or the like. According to some embodiments, the ruminant feed product may include at least one saturated fatty acid component containing at least about 70% of a palmitic acid compound by weight. The fatty acid component can generally be present in the ruminant feed product at any concentration. For example, some sample concentration ranges include about 5% by weight to about 80% by weight, about 10% by weight to about 70% by weight, and about 10% by weight to about 50% by weight. In some specific embodiments, the fatty acid component may be present in the ruminant feed product in an amount of about 5% by weight, about 10% by weight, about 15% by weight, about 20% by weight, about 25% by weight, about 30% by weight, about 40% by weight, about 50% by weight, or any value or range between any two of these values (including endpoints).

In some embodiments, the fatty acid component may have a melting point of at least about 40° C., about 80° C. or less, or about 40° C. to about 80° C. In particular embodiments, the fatty acid component may have a melting point of about 40° C., about 45° C., about 50° C., about 55° C., about 60° C., about 65° C., about 70° C., about 75° C., about 80° C., or any value or range between any two of these values (including endpoints). The melting point may be selected so that it is a temperature that aids in keeping the fatty acid inert in the rumen environment.

The palmitic acid compound is not limited by this disclosure, and may include one or more of a conjugated palmitic acid, unconjugated palmitic acid, free palmitic acid, palmitic acid derivatives, and/or the like. Palmitic acid, also known as hexadecanoic acid, has a molecular formula of CH₃(CH₂)₁₄CO₂H. Non-limiting examples of palmitic acid derivatives include palmitic acid esters, palmitic acid amides, palmitic acid salts, palmitic acid carbonates, palmitic acid carbamates, palmitic acid imides, palmitic acid anhydrides, and/or the like. According to some embodiments, the palmitic acid compound may include free palmitic acid, palmitate triglyceride, sodium palmitate, calcium palmitate, magnesium palmitate, ammonium palmitate, or combinations thereof.

The palmitic acid compound may be present in the saturated fatty acid component in an amount of about 60% by weight of the fatty acid to about 100% by weight of the fatty acid, including about 60% by weight, about 65% by weight, about 70% by weight, about 75% by weight, about 80% by weight, about 85% by weight, about 90% by weight, about 95% by weight, about 98% by weight, about 99% by weight, about 100% by weight, or any value or range between any two of these values (including endpoints). In an embodiment, the saturated fatty acid component may consist of 100% of the palmitic acid compound, in other words, the saturated fatty acid component is palmitic acid.

In some embodiments, the saturated fatty acid component may include a stearic acid compound. The stearic acid compound is not limited by this disclosure, and may include conjugated stearic acid, unconjugated stearic acid, free stearic acid, stearic acid derivatives, and/or the like. Stearic acid, also known as octadecanoic acid, has a molecular formula of CH₃(CH₂)₁₆CO₂H. Specific examples of stearic acid derivatives may include stearic acid esters, stearic acid amides, stearic acid salts, stearic acid carbonates, stearic acid carbamates, stearic acid imides, stearic acid anhydrides, and/or the like. Because stearic acid in large amounts may hinder milk production capacity of the mammary gland, the amount of stearic acid may be present in the fatty acid component in an amount of about 30% or less by weight of the fatty acid component. In particular embodiments, the stearic acid compound may include about 30% by weight of the fatty acid component, about 25% by weight of the fatty acid component, about 20% by weight of the fatty acid component, about 15% by weight of the fatty acid component, about 10% by weight of the fatty acid component, about 5% by weight of the fatty acid component, about 1% by weight of the fatty acid component, about 0% by weight of the fatty acid component, or any value or range between any two of these values. In some embodiments, the fatty acid component substantially does not contain a stearic acid compound.

According to some embodiments, the saturated fatty acid component may be free or substantially free of trans fatty acids. For example, a trans fatty acid component may be present in the ruminant feed product in an amount of about 5% by weight, about 3% by weight, about 2% by weight, about 1% by weight, about 0.5% by weight, about 0% by weight, or any value or range between any two of these values (including endpoints).

The ruminant feed product accessed 105 according to the method depicted in FIG. 1 may include other components, for instance, one or more nutrient components. The nutrient component may include, without limitation, carnitine, at least one glucogenic precursor, at least one vitamin, at least one mineral, at least one amino acid, at least one amino acid derivative, at least one antioxidant, at least one protein, or combinations thereof. The ruminant feed product may include various portions of the one or more nutrient components generally included in particular amounts that are sufficient to provide beneficial nutritional and dietary needs of the ruminant that is to consume the dietary composition. For example, the ruminant feed product may include a carbohydrate portion and a vitamin portion, each in an amount sufficient to provide beneficial nutritional and dietary needs of the ruminant.

In some embodiments, the feed ingredient may include an amount of carnitine. The carnitine may be included in the feed ingredient to aid in the breakdown of fatty acids to generate metabolic energy in the ruminant. In some embodiments, the carnitine may be present in a premix composition.

In various embodiments, the glucogenic precursor may include at least one of glycerol, propylene glycol, molasses, propionate, glycerine, propane diol, calcium propionate, propionic acid, octanoic acid, steam-exploded sawdust, steam-exploded wood chips, steam-exploded wheat straw, algae, algae meal, microalgae, or combinations thereof. The glucogenic precursor may generally be included in the feed ingredient to provide an energy source to the ruminant, for example, so as to prevent gluconeogenesis from occurring within the ruminant's body.

The antioxidant is not limited by this disclosure and may include any antioxidants or combination of antioxidants, particularly those used in the ruminant feed product. Illustrative examples of antioxidants may include alpha-carotene. beta-carotene, ethoxyquin, butylated hydroxyanisole (BHA), butylated hydroxytoluene (BHT), cryptoxanthin, lutein, lycopene, zeaxanthin, vitamin A, vitamin C, vitamin E, selenium, alpha-lipoic acid, and/or the like.

In various embodiments, the vitamin may include any combination of vitamins including, without limitation, vitamin A, vitamin B, vitamin D, vitamin E, vitamin C, vitamin K, and/or the like. Specific examples of vitamin B include thiamine (vitamin B₁), riboflavin (vitamin B₂), niacin (vitamin B₃), pantothenic acid (vitamin B₅), pyridoxine (vitamin B₆), biotin (vitamin B₇), folic acid (vitamin B₉), cobalamin (vitamin B₁₂), and choline (vitamin Bₚ).

In various embodiments, the amino acid may include any combination of common, uncommon, essential, and non-essential amino acids, including, without limitation, essential amino acids such as leucine, lysine, histidine, valine, arginine, threonine, isoleucine, phenylalanine, methionine, tryptophan, and/or any derivative thereof. In some embodiments, the amino acid may be a non-essential amino acid, including any combination of alanine, asparagine, aspartate, cysteine, glutamate, glutamine, glycine, proline, serine, tyrosine, and/or any derivative thereof. The amino acid and/or any derivative thereof may also include amino acids and derivatives of both non-essential and essential amino acids. The amino acid may generally be included in the feed ingredient to provide a nutritional aid in various physiological processes in the ruminant, such as, for example, increasing muscle mass, providing energy, aiding in recovery, and/or the like. In some embodiments, the amino acid may be present in a premix composition.

In various embodiments, the mineral may be any mineral that is a generally recognized as safe (GRAS) mineral or a combination of such minerals. The mineral may further be obtained from any mineral source that provides a bioavailable mineral. In some embodiments, the mineral may be one or more of calcium, sodium, magnesium, potassium, phosphorous, zinc, selenium, manganese, iron, cobalt, copper, iodine, molybdenum, and/or the like. In some embodiments, the mineral may be selected from one or more of a sodium salt, a calcium salt, a magnesium salt, a cobalt salt, a manganese salt, a potassium salt, an iron salt, a zinc salt, copper sulfate, copper oxide, selenium yeast, a chelated mineral, and/or the like. Illustrative examples of sodium salts include monosodium phosphate, sodium acetate, sodium chloride, sodium bicarbonate, disodium phosphate, sodium iodate, sodium iodide, sodium tripolyphosphate, sodium sulfate, sodium selenite, and/or the like. Illustrative examples of calcium salts include calcium acetate, calcium carbonate, calcium chloride, calcium gluconate, calcium hydroxide, calcium iodate, calcium iodobehenate, calcium oxide, anhydrous calcium sulfate, calcium sulfate dehydrate, dicalcium phosphate, monocalcium phosphate, tricalcium phosphate, and/or the like. Illustrative magnesium salts include magnesium acetate, magnesium carbonate, magnesium oxide, magnesium sulfate, and/or the like. Illustrative cobalt salts include cobalt acetate, cobalt carbonate, cobalt chloride, cobalt oxide, cobalt sulfate, and/or the like. Illustrative examples of manganese salts include manganese carbonate, manganese chloride, manganese citrate, manganese gluconate, manganese orthophosphate, manganese oxide, manganese phosphate, manganese sulfate, and/or the like. Illustrative examples of potassium salts include potassium acetate, potassium bicarbonate, potassium carbonate, potassium chloride, potassium iodate, potassium iodide, potassium sulfate, and/or the like. Illustrative examples of iron salts include iron ammonium citrate, iron carbonate, iron chloride, iron gluconate, iron oxide, iron phosphate, iron pyrophosphate, iron sulfate, reduced iron, and/or the like. Illustrative examples of zinc salts include zinc acetate, zinc carbonate, zinc chloride, zinc oxide, zinc sulfate, and/or the like.

In some embodiments, the protein used in the feed ingredient may be obtained from a protein source. Illustrative examples of protein sources may include one or more grains and/or oilseed meals. The grain is generally not limited by this disclosure and may be any edible grain, or combination of grains, that is used as a protein source. Illustrative examples of grains include cereal grains such as barley, millet, wheat, spelt wheat, rye, oats, triticale, rice, corn, buck wheat, quinoa, amaranthus, sorghum, and the like. Oilseed meal is generally derived from residue that remains after reserved oil is removed from oilseeds. The oilseed meal may be rich in protein and variable in residual fats and oils. Illustrative examples of oilseed meal includes rapeseed meal, soybean meal, sunflower meal, cottonseed meal, camelina meal, mustard seed meal, crambe seed meal, safflower meal, rice meal, peanut meal, corn gluten meal, corn gluten feed, distillers dried grains, distillers dried grains with solubles, wheat gluten, and/or the like. According to some embodiments, the ruminant feed product may include materials such as algae, algae meal, microalgae, or the like.

In some embodiments, the ruminant feed product may include at least one cellulosic material. The cellulosic material may generally provide a source of fiber for the ruminant to lower cholesterol levels and promote proper digestive function. Illustrative examples of cellulosic materials include wheat bran, wheat middlings, wheat mill run, oat hulls, oat bran, soya hulls, grass meal, hay meal, alfalfa meal, alfalfa, straw, hay, and/or the like.

In various embodiments, the ruminant feed product may include a micronutrient mixture. Micronutrient mixtures are not limited by this disclosure and may generally contain any micronutrient mixture now known or later developed. The micronutrient mixture may include various components, such as at least one vitamin and at least one mineral, as described in greater detail herein. In some embodiments, the micronutrient mixture may be present in a premix composition.

In various embodiments, the ruminant feed product or portions thereof may be subjected to a grinding process configured to form the feed ingredient or portions thereof into certain particle sizes and/or to achieve a more uniform particle size. For example, a carbohydrate and/or a protein component of the feed ingredient may be ground to a certain particle size. In another example, the feed ingredient itself may be ground to a certain particle size.

Grinding may be performed by various grinding devices known to those having ordinary skill in the art, such as a hammer mill, a roller mill, a disk mill, or the like. The feed ingredient and/or portions thereof may be ground to various sizes, such as particle size (for instance, measured in millimeters), mesh sizes, surface areas, or the like. According to some embodiments, the feed ingredient and/or portions thereof may be ground to a particle size of about 0.05 millimeters, about 1 millimeters, about 2 millimeters, about 5 millimeters, about 7 millimeters, about 10 millimeters, and values or ranges between any two of these values (including endpoints). In some embodiments, the various components may be ground so that about 20% to 50% of the each component and/or all components are retained by a mesh having openings with a size of about 10 mm and so that about 70% to about 90% of each component and/or all components are retained by a mesh having openings with a size of about 1 mm. In some embodiments, the various components may have a varying distribution of particle sizes based upon the ingredients. For example, in embodiments containing one or more wheat ingredients, the particle size may be distributed so that about 95% of the ground wheat ingredients are retained by a mesh having openings with a size of about 0.0625 mm and so that about 65% of the ground wheat ingredients are retained by a mesh having openings with a size of about 1.0 mm. In another example, such as embodiments containing one or more barley ingredients, the particle size may be distributed so that about 95% of the ground barley ingredients are retained by a mesh having openings with a size of about 0.0625 mm and so that about 60% of the ground barley ingredients are retained by a mesh having openings with a size of about 1.0 mm. The varying mesh sizes of each ingredient may be independent of mesh sizes for other ingredients.

Grinding may provide various benefits, such as improving certain characteristics of the feed ingredient and/or the dietary composition formed therefrom. For instance, even and fine particle size may improve the mixing of different ingredients. According to certain embodiments, grinding may be configured to decrease a particle size of certain components of the dietary composition, for example, to increase the surface area open for enzymes in the gastrointestinal tract, which may improve the digestibility of nutrients, and/or to increase the palatability of the feed.

In an embodiment, the ruminant feed product may include water. For example, the water content of the ruminant feed product may be about 5% by weight, about 10% by weight, about 12% by weight, about 15% by weight, about 25% by weight or any value or range between any two of these values (including endpoints). In an embodiment, the ruminant feed product may include a carbohydrate component and/or a nitrogen component. The carbohydrate component may generally include at least one of a sugar, a starch, or a grain. Non-limiting examples of carbohydrate components include cellulose, hemicellulose, sugar beet pulp, sugar cane, wheat bran, oat hulls, grain hulls, soybean hulls, peanut hulls, wood, brewery by-product, algae, algae meal, grasses, legumes, plant-based feedstuffs, wheat, corn, oats, sorghum, millet, and barley. Non-limiting examples of the nitrogen component include oilseed meals, soy meals, bean meals, rapeseed meals, sunflower meals, coconut meals, olive meals, linseed meals, and grapeseed meals. The ruminant feed product may include various concentrations of the carbohydrate component and/or the nitrogen component. For example, the carbohydrate component content of the ruminant feed product may be about 0.1% to about 55% by weight and the nitrogen component of the ruminant feed product may be about 0.1% to about 55% by weight. In an embodiment, the ruminant feed product may include a micellizing agent. Non-limiting examples of micellizing agents may include lecithin, cephalin, castor oil ethoxylate, sorbitan monooleate, tallow ethoxylate, lauric acid, polyethylene glycol, or combinations thereof.

Referring to FIG. 1, at least one physical characteristic of the ruminant may be determined 110. The at least one physical characteristic may include, without limitation, the age, weight, pedigree or heritage, genetics, physical condition, health condition, milk yield, milk fat content, or the like. According to some embodiments, the at least one physical characteristic may be determined 110 at various times, such as periodically (for instance, daily, weekly, monthly, or the like) or according to various triggers, such as a noticeable change in a characteristic of the ruminant (for instance, an illness, a calving event, or the like). At least one time period associated with the ruminant may be determined 115. The at least one time period may include, without limitation, age, calving cycle, life stage, illness progression, season, lactation cycle, or the like. The at least one physical characteristic of the ruminant and the at least one time period associated with the ruminant may be determined 110, 115 and used as an indicator of the physical condition and/or the dietary needs of the ruminant.

Lactation cycles have various “periods” defined relative to the calving event. For example, in dairy cows, the “dry cow period” is the period from about eight or six weeks before calving until calving. In general, milk product may begin at calving and may end by at or just before the dry period. The dry cow period is subdivided into the “far-off dry cow period”, which is from about eight or six weeks before calving to three weeks before calving, and the “close-up dry cow period”, which is from about three weeks before calving until calving. The period from calving until about three weeks after calving is called the “fresh cow period.” Lactation (milk production) occurs during and after the fresh cow period. “Peak lactation” typically occurs sometime around six to nine weeks after calving. After peak lactation, there is a “constant phase” of relatively constant milk production, followed by “late lactation” during which milk production gradually decreases.

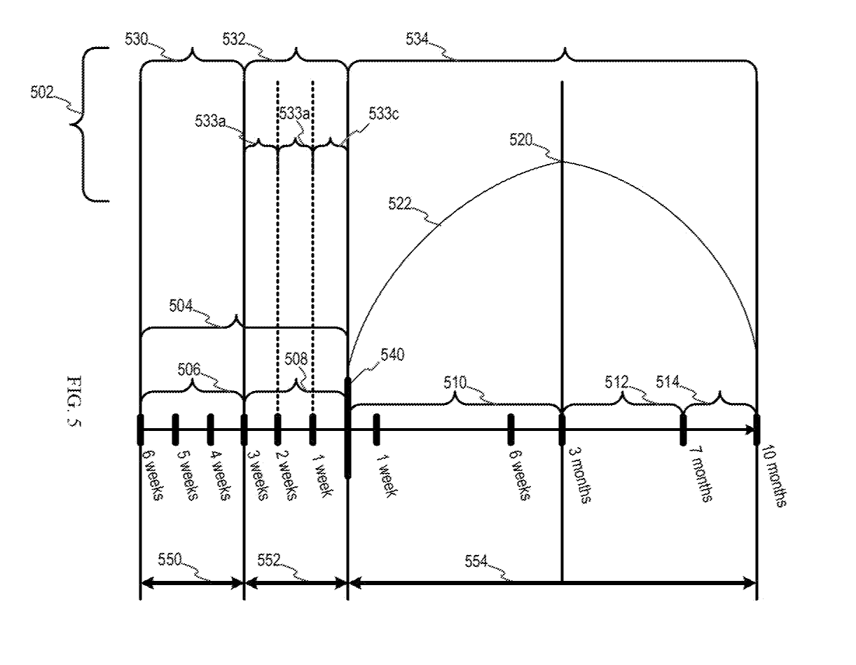

Ruminants, such as dairy cows, typically eat more food after calving than before calving. For example, in the dry cow period, a dairy cow might eat about 1.5% to about 2.5% by weight of dry matter. For example, the dairy cow might eat about 2% of their body weight in dry matter per day. After calving, the dairy cow gradually increases food intake until the cow is eating about 4% to about 7% by weight of dry matter. For example, the cow might eat about 5% of their body weight in dry matter per day. Dry matter includes any solid state feed materials that the cow ingests including, for example, pellets feed, loose particulate feed, or total mixed ration (TMR) feed. In general, TMR is a mix of grain and silage with some protein meals, such as, for example, soya bean meal and canola meal. Additional materials and trace elements, vitamins, extra nutrients, and the like may also be added to the TMR. Refer to FIG. 5 for an example illustration of a lactation cycle and a feed program for a ruminant during various phases of the lactation cycle according to some embodiments described herein.

In some general embodiments, various periods or sub-periods within the lactation cycle can be used to vary the amount of a ruminant feed product or the amount of saturated fatty acid fed to the ruminant. For example, a first level C1 can be fed to the ruminant during a first time period P1, a second level C2 can be fed to the ruminant during a second time period P2, and a third level C3 can be fed to the ruminant during a third time period P3. The second level C2 may be higher than the first level C1. The level may be the concentration or amount. The level may gradually increase from C1 to C2, or may increase in one or more steps.

In some embodiments, the first time period P1 may include the far-off dry cow period. For example, P1 can be from about eight to six weeks before calving to about calving. In one embodiment, P1 can be from about eight weeks before calving to about calving. In another embodiment, P1 can be from about six weeks before calving to about calving. In some embodiments, C1, as the concentration or amount of the grams of the palmitic acid compound per kilogram of body weight (“grams/kilogram” or “g/kg”), can be at most 0.04 grams/kilogram. In some embodiment, C1 can be less than 0.02 grams/kilogram. In some embodiments, substantially no or no palmitic acid compound is fed to the ruminant during P1.

In some embodiments, the second time period P2 may include the close-up dry cow period. For example P2 can be about three weeks before calving to about calving.

The second time period P2 can be further divided into multiple sub-periods such as P2a, P2b, and P2c. For example, when P2 is the three-week long period before calving, sub-period P2a can be about three weeks before calving to about two weeks before calving, P2b can be about two weeks before calving to about one week before calving, and P2c can be about one week before calving to calving (for instance, less than a week before calving to about less than a week after calving). Concentration or amount C2a can be fed to the ruminant during P2a, concentration or amount C2b can be fed to the ruminant during P2b, and concentration or amount C2c can be fed to the ruminant during P2c. Concentration or amount can be the grams of a saturated fatty acid (such as palmitic acid) per kilogram of body weight (“grams/kilogram” or “g/kg”). For example, C2a can be about 0.04 grams/kilogram to about 0.08 grams/kilogram body weight. For example, C2b can be about 0.08 grams/kilogram to about 0.16 grams/kilogram body weight. For example, C2c can be about 0.14 grams/kilogram to about 0.22 grams/kilogram body weight.

In some embodiments, the third time period P3 may include at least one of the fresh cow period, the peak lactation period, the constant phase of lactation period, and the late lactation phase, or any combinations. For example, P3 can be from calving to about 48 weeks after calving. In yet other embodiments, P3 can include all or a portion of the fresh cow period, the peak lactation period, the constant phase of lactation period, and the late lactation phase. For example, P3 can be from about three days after calving to about 48 weeks after calving. Alternatively, P3 can be from about calving to about nine weeks after calving. Alternatively, P3 can be about one week after calving to about 24 weeks after calving. Alternatively, P3 can be about one week after calving to about 36 weeks after calving. Alternatively, P3 can be about one week after calving to about 42 weeks after calving. In some embodiments, concentration or amount C3 is greater than C2. In some embodiments, when the concentration or amount is the grams of a saturated fatty acid (such as palmitic acid) per kg of body weight, C3 may be about 2, about 3, about 4, about 5, about 6, about 7, about 8, about 9, or about 10 times C2.

In some embodiments, the time period P3 can be after the fresh cow period. For example, P3 can be about three weeks or more after calving until the far-off dry cow period. In some embodiments, concentration or amount C3 is defined by grams of palmitic acid compound per kilogram of milk yield. For example, C3 can be about 5 grams/kilograms, about 7.5 grams/kilograms, about 10 grams/kilograms, about 12.5 grams/kilograms, about 15 grams/kilograms, or ranges between any two of these values (including endpoints).

The ruminant may be fed 120 an amount of the ruminant feed product based on the at least one physical characteristic and the at least one time period. For example, the ruminant may be fed a certain amount of the ruminant feed product, the saturated fatty acid compound, or the palmitic acid compound based on the weight and the age of the ruminant, such as X grams of the ruminant feed product for every Y kilograms of weight of the ruminant between age T and age U, 2X grams of the ruminant feed product for every Y kilograms of weight of the ruminant between age V and age W, and so on. In another example, the amount of the ruminant feed product, the saturated fatty acid compound, or the palmitic acid compound may be increased or decreased while the ruminant is experiencing an illness or during certain seasons (for instance, the ruminants may require more energy during certain seasons as compared to other seasons). Feeding the ruminant amounts of the ruminant feed product based on the physical and/or dietary needs of the ruminant as determined by a time period associated with the ruminant may improve the milk yield and/or fat content of the milk The physical and/or dietary needs may include, without limitation, energy needs, growth needs, milk production needs, or the like.

According to some embodiments, different physical characteristics of the ruminant may be more indicative of the amount of the ruminant feed product that may be fed to the ruminant to improve milk yield and/or fat content at different time periods associated with the ruminant. As such, the amount of the ruminant feed product fed 120 to the ruminant may be based on different physical characteristics at different time periods. For example, the weight of the ruminant may be used during a first time period, the age of the ruminant may be used during a second time period, the milk yield of the ruminant may be used during a third time period, and so on. According to some embodiments, the diet of the ruminant may consist or consist essentially of only the ruminant feed product with the exception of, for example, medicine, supplements, or the like.

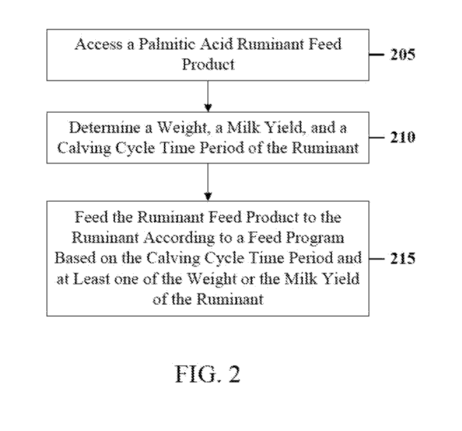

FIG. 2 depicts a method for feeding a ruminant according to a second embodiment. As shown in FIG. 2, a palmitic acid ruminant feed product may be accessed 205 for feeding to the ruminant. Characteristics of the ruminant including the weight, milk yield and calving cycle time period associated with the ruminant may be determined 210. According to some embodiments, the calving cycle time period may be configured as a certain amount of time before or after the ruminant is scheduled to calve or has calved. Non-limiting examples of calving cycle time periods include 4 weeks before calving, 3 weeks before calving, 2 weeks before calving, 1 week before calving, 1 week after calving, 2 weeks after calving, 3 weeks after calving, 4 weeks after calving, and a non-calving period (for instance, 5 or more weeks after calving), and values or ranges between any two of these values (including endpoints). As described herein, the calving cycle time periods may be described in terms of time periods, including, without limitation, the dry cow period, the far-off dry cow period, the close-up dry cow period, the fresh cow period, peak lactation, constant phase, late lactation, or the like.

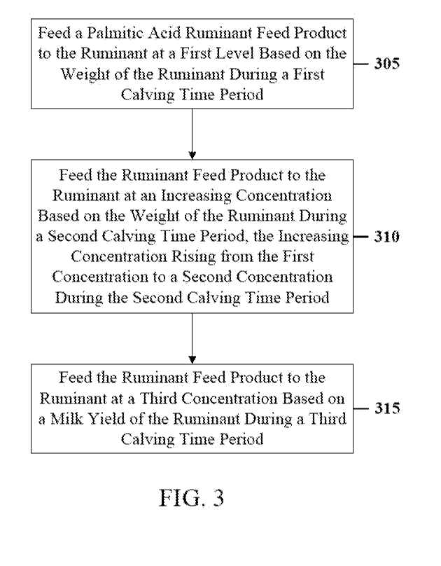

The calving cycle time period may be used to indicate the physical and/or dietary needs of the ruminant. The ruminant may be fed 215 according to a feed program configured based on the calving cycle time period and at least one of the weight or the milk yield of the ruminant (see FIG. 3, below, for an illustrative feed program according to some embodiments). The feed program may be used to feed 215 the ruminant in a manner that improves the milk yield and/or milk fat content of milk, including the colostrum, produced by the ruminant in view of the physical and/or dietary needs of the ruminant as the ruminant progresses through the calving cycle.

According to some embodiments, a system, such as an automatic cattle feeding system, may be used to perform various steps of the methods described herein. The system may include various components, including, without limitation, a ruminant feed product source, elements for accessing the ruminant feed product from the ruminant feed product source, ruminant feed product containers (for example, troughs, lick tanks, individual feed containers, or the like), and elements for measuring and/or dispensing the ruminant feed product. In an embodiment, the system may include ruminant identification devices configured to identify at least one ruminant and to adjust a feed program to correspond with the at least one ruminant. For example, the system may be configured to dispense an amount of the ruminant feed product to a particular ruminant based on the physical characteristics of the ruminant, a time period associated with the ruminant, and the concentration of the ruminant feed product based on the physical characteristics and the time period.

FIG. 3 depicts an illustrative feed program according to a first embodiment. As shown in FIG. 3, a feed product may be fed 305 to the ruminant at a first concentration based on the weight of the ruminant during a first calving time period (or “first time period”). The feed product may or may not include palmitic acid. In various embodiments, the first calving time period may include from about 9 weeks, 8 weeks, 7 weeks, or 6 weeks before calving to about 3 weeks before calving, The palmitic acid ruminant feed product may include a feed product including a fatty acid component, such as a palmitic acid component. Accordingly, the amount, concentration, level or other measure of the fatty acid component may be adjusted as described herein to provide the ruminant with a concentration, level, amount, or the like of the fatty acid component. The remainder of the ruminant feed product, for instance, the portion of the ruminant feed product to be fed to the ruminant that is not the fatty acid component, may include dry matter and other materials described herein. According to some embodiments, the first level may be less than 0.04 grams of palmitic acid per kilogram of body weight (“grams/kilograms” or “g/kg”). According to some embodiments, the first level may be substantially free of palmitic acid.

The ruminant may be fed 310 the fatty acid component at an increasing concentration based on the weight of the ruminant during a second calving time period (or “second time period”). In various embodiments, the second calving time period may be from about 3 weeks before calving, about 2 weeks before calving, about 1 week before calving, to calving. According to some embodiments, the second level may include about 0.04 grams of palmitic acid per kilogram of body weight (“grams/kilograms”), about 0.08 grams of palmitic acid per kilogram of body weight, about 0.16 grams of palmitic acid per kilogram of body weight, about 0.22 grams of palmitic acid per kilogram of body weight, or values or ranges between any two of these values (including endpoints). For instance, for a ruminant having a body weight of about 750 kilograms, the amount of palmitic acid fed to the ruminant may be about 60 grams, about 127.5 grams, about 255 grams, about 375 grams, or values or ranges between any two of these values (including endpoints). In an embodiment, the second level may be determined based on feeding a certain number of grams (for instance, about 50 grams to about 100 grams) of palmitic acid or the fatty acid component per 600 kilograms of body weight of the ruminant.

The palmitic acid ruminant feed product may be fed 315 to the ruminant at a third level during a third calving time period (or “third time period”). The third level may be based on palmitic acid grams per kilogram of body weight or palmitic acid grams per kilogram of milk yield by the ruminant In various embodiments, the third calving time period may include from calving to about 1 week after calving, about 2 weeks after calving, about 3 week after calving, about 5 weeks, about 9 weeks, about 20 weeks, about 40 weeks or more after calving, or values or ranges between any two of these values (including endpoints). According to some embodiments, the third level may be at least 0.4 gram palmitic acid per kilogram of body weight from about calving to about 1 week after calving. According to some embodiments, the third level may include about 2 grams of the fatty acid component per kilogram of milk yield, about 5 grams of the fatty acid component per kilogram of milk yield, about 10 grams of the fatty acid component per kilogram of milk yield, about 20 grams of ruminant feed product per kilogram of milk yield, or values or ranges between any two of these values (including endpoints), from one week after calving to until the far-off dry cow period. The third calving time period may include all or a portion of post-calving time period for the ruminant up to the far-off dry cow period. In an embodiment, feeding 315 the palmitic acid ruminant feed product to the ruminant at an increasing concentration based on the weight of the ruminant during the third calving time period may allow the ruminant to produce about 7 kilograms to about 10 kilograms of colostrum in a first milking after calving.

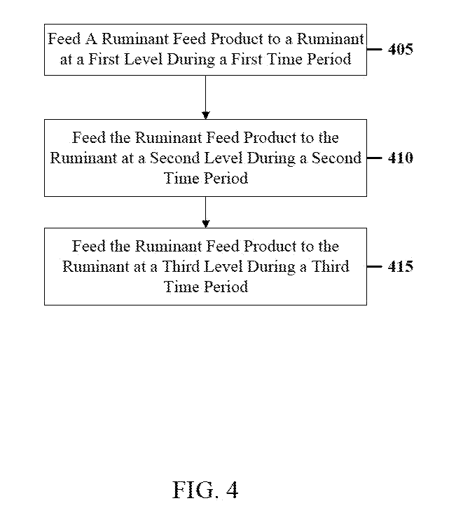

FIG. 4 depicts an illustrative feed program according to a first embodiment. As shown in FIG. 4, a ruminant feed product may be fed 405 to a ruminant at a first level during a first time period. According to some embodiments, the first level include a particular amount or concentration of the fatty acid component and/or ruminant feed dry matter of the ruminant feed product. In an embodiment, the first level may be free or substantially free of the saturated fatty acid component. In various embodiments, the fatty acid component in the first level may not exceed about 0.01 grams per kilogram of the body weight of the ruminant (“grams/kilogram”), about 0.02 grams/kilogram, about 0.04 grams/kilogram, about 0.06 grams/kilogram, and ranges and values between any two of these values (including endpoints). In another embodiment, the first level may include ruminant dry matter at about 1.0% of the body weight of the ruminant, about 1.5% of the body weight of the ruminant, about 2.0% of the body weight of the ruminant, about 2.5% of the body weight of the ruminant, and ranges and values between any two of these values (including endpoints).

In an embodiment, the first time period may include a dry cow period, such as a far-off dry cow period. In various embodiments, the first time period may include about 9 weeks, about 8 weeks, about 7 weeks, about 6 weeks, about 5 weeks, about 4 weeks, or about 3 weeks before calving to about calving.

The ruminant feed product may be fed 410 to the ruminant at a second level during a second time period. In an embodiment, the fatty acid component in the second level may include at least about 0.04 grams/kilogram, about 0.06 grams/kilogram, about 0.08 grams/kilogram, about 0.10 grams/kilogram, about 0.20 grams/kilogram, about 0.30 grams/kilogram, about 0.40 grams/kilogram and ranges and values between any two of these values (including endpoints). In an embodiment, the second time period may include a dry cow period, such as a close-up dry cow period. In various embodiments, the second time period may include about 4 weeks before calving, about 3 weeks before calving, about 2 weeks before calving, about 1 week before calving, a calving time period (for instance, less than a week before calving to about less than a week after calving), and ranges and values between any two of these values (including endpoints).

In an embodiment, the second level may include various concentrations of the ruminant feed product and/or components thereof during various sub-time periods of the second time period. For example, the second level may include a first concentration during a first sub-time period, a second concentration during a second sub-time period, a third concentration during a third sub-time period, and so on. In an embodiment, the first sub-time period may include about 3 weeks before calving, the second sub-time period may include about 2 weeks before calving, and the third sub-time period may include about 1 week before calving.

In various embodiments, the first concentration may be about 0.01 gram/kilogram, about 0.02 grams/kilogram, about 0.04 grams/kilogram, about 0.05 grams/kilogram, about 0.06 grams/kilogram, about 0.08 grams/kilogram, about 0.10 grams/kilogram, and ranges and values between any two of these values (including endpoints). In various embodiments, the second concentration may be about 0.02 gram/kilogram, about 0.04 grams/kilogram, about 0.06 grams/kilogram, about 0.08 grams/kilogram, about 0.10 grams/kilogram, about 0.12 grams/kilogram, about 0.14 grams/kilogram, about 0.16 grams/kilogram, about 0.20 grams/kilogram and ranges and values between any two of these values (including endpoints). In various embodiments, the third concentration may be about 0.02 gram/kilogram, about 0.04 grams/kilogram, about 0.08 grams/kilogram, about 0.10 grams/kilogram, about 0.14 grams/kilogram, about 0.18 grams/kilogram, about 0.20 grams/kilogram, about 0.22 grams/kilogram, about 0.26 grams/kilogram and ranges and values between any two of these values (including endpoints).

A ruminant feed product may be fed 415 to the ruminant at a third level during a third time period. In various embodiments, the fatty acid component in the third level may include at least about 0.4 grams/kilogram, about 0.5 grams/kilogram, about 0.6 grams/kilogram, about 0.8 grams/kilogram, about 1.0 grams/kilogram, about 1.2 grams/kilogram, about 1.5 grams/kilogram, and ranges and values between any two of these values (including endpoints). In an embodiment, the fatty acid component in the third level may include at most about 0.5 grams/kilogram. In another embodiment, the third level may include ruminant dry matter at about 2% of the body weight of the ruminant, about 4% of the body weight of the ruminant, about 6% of the body weight of the ruminant, about 7% of the body weight of the ruminant, about 10% of the body weight of the ruminant, and ranges and values between any two of these values (including endpoints).

According to some embodiments, the third time period may include various lactation periods, such as a fresh cow period, a peak lactation period, a constant phase of lactation period, and a late lactation period. In various embodiments, the third time period may include a calving time period (for instance, less than a week before calving to about less than a week after calving) to a far-off dry cow period.

The third level of the ruminant feed product fed 415 may vary according to the particular third time period. In an embodiment, the third level may include one of the following during the first week after calving: about 0.4 grams to about 0.8 grams/kilogram; at most 0.5 grams/kilogram during the first week after calving; about 10 grams of the fatty acid component per kilogram of the milk yield. In an embodiment, the third level may include at least 0.4 grams/kilogram during the fresh cow period. In various embodiments, the third level may be at least about 8 grams of the fatty acid component per kilogram of the milk yield, about 10 grams of the fatty acid component per kilogram of the milk yield, about 12 grams of the fatty acid component per kilogram of the milk yield, about 16 grams of the fatty acid component per kilogram of the milk yield, and ranges and values between any two of these values (including endpoints) during the peak lactation period, the constant phase of lactation, and/or the late lactation period.

FIG. 5 depicts an illustrative lactation cycle for a ruminant (for instance, a dairy cow) and method for feeding the ruminant according to a feed program configured according to some embodiments. As shown in FIG. 5, the lactation cycle of a dairy cow may include various time periods that may be measured relative to a calving 540 event. For example, a dry cow period 504 may extend from about 8 or 6 weeks before calving 540 to calving. The dry cow period 504 may include a far-off dry cow period 506, about 8 or 6 weeks before calving 540 to about 3 weeks before calving, and a close-up dry cow period 508, about 3 weeks before calving to about calving. A fresh cow period 510 may extend from about calving 540 to about 8 weeks after calving. A constant phase of lactation period 512 may extend for about 3 weeks to about 4 weeks from the end of the fresh cow period 510. A 100-day late lactation period 514 may extend up until the dry cow period 504 for the ruminant. The dry cow period 504 may follow the 100-day late lactation period 514 as the lactation cycle repeats for the ruminant (for instance, if the ruminant calves again, such as once a year (about 350 days to about 425 days)). A lactation curve 520 graphically represents the lactation cycle for a ruminant over the various lactation periods 504-514, including a peak lactation 522 that may generally occur between the end of the fresh cow period 510 and the beginning of the constant phase of lactation 512.

According to some embodiments, the various lactation periods 504-514 may be segmented into time periods 550-554 for feeding the dairy cow at a particular level 530-534 during the lactation cycle. For example, a first time period 550 may extend through the far-off dry cow period 506 and may include feeding the dairy cow at a first level 530, which may be about 0 grams of a fatty acid component per kilogram of body weight of the dairy cow (“grams/kilograms”) to at most 0.4 grams/kilogram. As shown in FIG. 5, the fatty acid component may include a palmitic acid component of a ruminant feed product. A second time period 552 may extend through the close-up dry cow period 508 and may include feeding the dairy cow at a second level 532 of about 0.04 grams/kilogram to about 0.12 grams/kilogram.

The second level 532 may be segmented into multiple concentrations 533a-533c. The concentrations may include a first concentration 533a of about 0.04 grams/kilogram to about 0.08 grams/kilogram, a second concentration 533a of about 0.08 grams/kilogram to about 0.16 grams/kilogram, and a third concentration 533a of about 0.14 grams/kilogram to about 0.22 grams/kilogram.

A third time period 554 may start after calving 540 and may include a third level 534 in which the dairy cow is fed about 0.4 grams/kilogram to about 0.5 grams/kilogram from calving to about one week after calving, then 10 grams of the fatty acid component per kilogram of milk for the dairy cow.

In various embodiments, methods for increasing milk fat content in milk produced by ruminants may include providing a ruminant feed product as described herein to the ruminant for ingestion. In some embodiments, providing the dietary composition to the ruminant for the ruminant to consume may result in an increase in production of milk and/or an increase in fat content of the milk produced. These increases may generally be relative to a similar ruminant that does not receive the dietary composition according to feed methods described herein, an average of similar ruminants not receiving the dietary composition according to feed methods described herein, an average of the milk production quantity and fat content of the same ruminant when not provided the dietary composition according to feed methods described herein, and/or the like. In particular embodiments, the milk production (or milk yield) may increase by an amount of about 1% to about 10%, including about 1%, about 2%, about 3%, about 4%, about 5%, about 6%, about 7%, about 8%, about 9%, about 10%, or any value or range between any two of these values (including endpoints) as compared to milk production in dairy cows that do not ingest the ruminant feed product according to the methods described herein. In particular embodiments, the milk fat content may increase by an amount of about 10% to about 15%, including about 10%, about 11%, about 12%, about 13%, about 14%, about 15%, or any value or range between any two of these values (including endpoints) as compared to the fat content of milk produced by dairy cows that do not ingest the ruminant feed product according to the methods described herein.

### EXAMPLES

### Example 1

### Feeding a Dairy Cow During a Calving Cycle

A dairy cow is provided with a ruminant feed product prepared to increase the milk fat and the quantity of the milk produced according to a feed program. The ruminant feed product includes a saturated fatty acid component that includes about 90% free palmitic acid, wheat, barley, and sunflower meals. The ruminant feed product also includes Vitamin A and an arginine derivative as nutrient components. The ruminant feed product is in a pellet form. The ruminant has a body weight of about 700 kilograms.

On Jan. 1, 2013, the dairy cow is about 3 weeks before calving (expected calving date of Jan. 22, 2013). The dairy cow is fed about 0.20 grams of the palmitic acid per kilogram of body weight (“grams/kilograms”) from a time period starting about 3 weeks before (Jan. 1, 2013) calving until about 1 week before the dairy cow calves (Jan. 15, 2013). Accordingly, from about Jan. 1, 2013 to about Jan. 15, 2013, the dairy cow is fed about 140 grams of the palmitic acid per day.

From about one week before calving (Jan. 16, 2013) until about 1 week after calving (Jan. 29, 2013), the dairy cow is fed an increasing concentration of palmitic acid. The increasing concentration starts at about 0.20 grams/kilograms and ends at about 0.50 grams/kilograms over the time period of about two weeks spanning from Jan. 16, 2013 until Jan. 29, 2013 (13 days). The concentration increase has an increase step of one day such that the concentration of the ruminant feed product increases by (0.50 grams/kilograms−0.20 grams/kilograms)/13 days≈0.023 grams/kilograms per day. Accordingly, on Jan. 16, 2013, the dairy cow is fed about 140 grams of the ruminant feed product, increasing by about 0.023 grams/kilograms per day or about 16.1 grams per day, to about 350 grams of the ruminant feed product on Jan. 29, 2013.

After Jan. 29, 2013, about 1 week after calving, the dairy cow is in a post-calving or non-calving state and the ruminant is fed based on the milk yield of the ruminant. The concentration of palmitic acid fed to the dairy cow is about 10 grams per kilogram of milk yield per day. On Jan. 30, 2013, the dairy cow has a milk yield of about 40 kilograms of milk per day and, accordingly, is fed about 400 grams of palmitic acid.

As a result of being fed according to the feed program, the dairy cow produces about 10% more milk containing about 10% more milk fat content than when on a diet that did not consist of the ruminant feed product.

### Example 2

### Dairy Cow Feeding System

An automated cattle feeding system (the “system”) may be configured to feed a plurality of dairy cows a ruminant feed product. The system may include a container for each of the dairy cows and a dispenser to dispense an amount of the ruminant feed product into the container. The ruminant feed product may include a saturated fatty acid component that has a palmitic acid compound content of about 95% and a stearic acid compound content of about 5%. The saturated fatty acid component is substantially free of trans fatty acids. The ruminant feed product includes a carbohydrate component of sugar cane, millet, and barley, a nitrogen component of sunflower meals and linseed meals, and a nutrient component of vitamin B and a mineral of calcium.

The system is configured to receive information indicating a body weight, milk yield, and a calving cycle time period associated with each of the dairy cows. The system is also configured to access a feed program for feeding the dairy cows according to the calving cycle time period and one of the body weight or the milk yield. The dispenser deposits an amount of the ruminant feed product into each container based on the feed program.

The feed program is configured such that a dairy cow is fed (1) about 0.30 grams of palmitic acid per kilogram of body weight (“grams/kilograms”) per day from about 2 weeks before calving to about 1 week before calving, (2) about 0.30 grams/kilograms per day from about 1 week before calving to about 0.6 grams/kilograms per day at about 2 weeks after calving, increasing about 0.014 grams/kilograms per day, and (3) about 15 grams per kilogram of milk yield per day after about 2 weeks after calving.

A first container is used by a dairy cow having a milk yield of about 35 kilograms per day that is about 5 weeks after calving. The system dispenses about (35)(15)=525 grams of the palmitic acid at the first container. A second container is used by a dairy cow having a body weight of about 800 kilograms that is about 2 weeks before calving. The system dispenses about (0.3)(800)=240 grams of palmitic acid at the second container.

Consumption of the ruminant feed product by the dairy cows results in a daily milk yield increase of about 7% and a milk fat content increase of about 8% compared to dairy cows that do not ingest the palmitic acid ruminant feed product pellets according to the feed program.

In the above detailed description, reference is made to the accompanying drawings, which form a part hereof. In the drawings, similar symbols typically identify similar components, unless context dictates otherwise. The illustrative embodiments described in the detailed description, drawings, and claims are not meant to be limiting. Other embodiments may be used, and other changes may be made, without departing from the spirit or scope of the subject matter presented herein. It will be readily understood that the aspects of the present disclosure, as generally described herein, and illustrated in the Figures, can be arranged, substituted, combined, separated, and designed in a wide variety of different configurations, all of which are explicitly contemplated herein.

The present disclosure is not to be limited in terms of the particular embodiments described in this application, which are intended as illustrations of various aspects. Many modifications and variations can be made without departing from its spirit and scope, as will be apparent to those skilled in the art. Functionally equivalent methods and apparatuses within the scope of the disclosure, in addition to those enumerated herein, will be apparent to those skilled in the art from the foregoing descriptions. Such modifications and variations are intended to fall within the scope of the appended claims. The present disclosure is to be limited only by the terms of the appended claims, along with the full scope of equivalents to which such claims are entitled. It is to be understood that this disclosure is not limited to particular methods, reagents, compounds, compositions or biological systems, which can, of course, vary. It is also to be understood that the terminology used herein is for the purpose of describing particular embodiments only, and is not intended to be limiting.

With respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity.

It will be understood by those within the art that, in general, terms used herein, and especially in the appended claims (for example, bodies of the appended claims) are generally intended as “open” terms (for example, the term “including” should be interpreted as “including but not limited to,” the term “having” should be interpreted as “having at least,” the term “includes” should be interpreted as “includes but is not limited to,” et cetera). While various compositions, methods, and devices are described in terms of “comprising” various components or steps (interpreted as meaning “including, but not limited to”), the compositions, methods, and devices can also “consist essentially of” or “consist of” the various components and steps, and such terminology should be interpreted as defining essentially closed-member groups. It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to embodiments containing only one such recitation, even when the same claim includes the introductory phrases “one or more” or “at least one” and indefinite articles such as “a” or “an” (for example, “a” and/or “an” should be interpreted to mean “at least one” or “one or more”); the same holds true for the use of definite articles used to introduce claim recitations. In addition, even if a specific number of an introduced claim recitation is explicitly recited, those skilled in the art will recognize that such recitation should be interpreted to mean at least the recited number (for example, the bare recitation of “two recitations,” without other modifiers, means at least two recitations, or two or more recitations). Furthermore, in those instances where a convention analogous to “at least one of A, B, and C, et cetera” is used, in general such a construction is intended in the sense one having skill in the art would understand the convention (for example, “a system having at least one of A, B, and C” would include but not be limited to systems that have A alone, B alone, C alone, A and B together, A and C together, B and C together, and/or A, B, and C together, et cetera). In those instances where a convention analogous to “at least one of A, B, or C, et cetera” is used, in general such a construction is intended in the sense one having skill in the art would understand the convention (for example, “a system having at least one of A, B, or C” would include but not be limited to systems that have A alone, B alone, C alone, A and B together, A and C together, B and C together, and/or A, B, and C together, et cetera). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, such as in terms of providing a written description, all ranges disclosed herein also encompass any and all possible subranges and combinations of subranges thereof. Any listed range can be easily recognized as sufficiently describing and enabling the same range being broken down into at least equal halves, thirds, quarters, fifths, tenths, et cetera As a non-limiting example, each range discussed herein can be readily broken down into a lower third, middle third and upper third, et cetera As will also be understood by one skilled in the art all language such as “up to,” “at least,” and the like include the number recited and refer to ranges which can be subsequently broken down into subranges as discussed above. Finally, as will be understood by one skilled in the art, a range includes each individual member. Thus, for example, a group having 1-3 cells refers to groups having 1, 2, or 3 cells. Similarly, a group having 1-5 cells refers to groups having 1, 2, 3, 4, or 5 cells, and so forth.

Various of the above-disclosed and other features and functions, or alternatives thereof, may be combined into many other different systems or applications. Various presently unforeseen or unanticipated alternatives, modifications, variations or improvements therein may be subsequently made by those skilled in the art, each of which is also intended to be encompassed by the disclosed embodiments.

What is claimed is:

1. A method for feeding a ruminant, the method comprising:
determining a body weight of the ruminant and a milk yield of the ruminant; and
providing a ruminant feed product to the ruminant for ingestion according to a feed program;
wherein the ruminant feed product comprises at least one fatty acid component comprising at least about 70% of a palmitic acid compound by weight;
wherein the feed program comprises:
feeding the ruminant feed product to the ruminant at a first level during a first time period, wherein the first level does not exceed about 0.04 grams of the fatty acid component per kilogram of the body weight,
feeding the ruminant feed product to the ruminant at a second level during a second time period, wherein the second level comprises at least 0.04 grams of the fatty acid component per kilogram of the body weight, and
feeding the ruminant feed product to the ruminant at a third level during a third time period, wherein the third level comprises at least 0.4 grams of the fatty acid component per kilogram of the body weight.

2. The method of claim 1, wherein the first time period comprises a far-off dry cow period.

3. The method of claim 1, wherein the first time period comprises from about 6 weeks before calving to about 3 weeks before calving.

4. The method of claim 1, wherein the first level comprises a ruminant feed dry matter from about 1.5% to about 2.5% of the body weight.

5. The method of claim 1, wherein the first level is substantially free of saturated fatty acid component.

6. The method of claim 1, wherein the first level does not exceed 0.02 grams of the fatty acid component per kilogram of the body weight.

7. The method of claim 1, wherein the second time period comprises a close-up dry cow period.

8. The method of claim 1, wherein the second time period is about 3 weeks before calving to about calving.

9. The method of claim 8, wherein the second time period comprises a third week before calving, a second week before calving, and a first week before calving.

10. The method of claim 9, wherein the second level comprises a first concentration during the third week before calving, a second concentration during the second week before calving, and a third concentration during the first week before calving.

11. The method of claim 10, wherein the first concentration comprises at least 0.04 grams of the fatty acid component per kilogram of the body weight.

12. The method of claim 10, wherein the first concentration comprises from about 0.04 grams to about 0.08 grams of the fatty acid component per kilogram of the body weight.

13. The method of claim 10, wherein the first concentration comprises about 0.05 grams of the fatty acid component per kilogram of the body weight.

14. The method of claim 10, wherein the second concentration comprises at least 0.08 grams of the fatty acid component per kilogram of the body weight.

15. The method of claim 10, wherein the second concentration comprises from about 0.08 to about 0.16 grams of the fatty acid component per kilogram of the body weight.

16. The method of claim 10, wherein the second concentration comprises about 0.12 grams of the fatty acid component per kilogram of the body weight.

17. The method of claim 10, wherein the third concentration comprises at least 0.14 grams of the fatty acid component per kilogram of the body weight.

18. The method of claim 10, wherein the third concentration comprises from about 0.14 grams to about 0.22 grams of the fatty acid component per kilogram of the body weight.

19. The method of claim 10, wherein the third concentration comprises about 0.18 grams of the fatty acid component per kilogram of the body weight.

20. The method of claim 1, wherein the third time period comprises one or more of a fresh cow period, a peak lactation period, a constant phase of lactation period, and a late lactation period.

21. The method of claim 1, wherein the third time period is from about calving to a far-off dry cow period.

22. The method of claim 1, wherein the third level comprises a ruminant feed dry matter from about 4% to about 7% of the body weight.

23. The method of claim 1, wherein the third level comprises from about 0.4 grams to about 0.8 grams of the fatty acid component per kilogram of the body weight during the first week after calving.

24. The method of claim 1, wherein the third level comprises at most 0.5 grams of the fatty acid component per kilogram of the body weight during the first week after calving.

25. The method of claim 1, wherein the third level comprises about 10 grams of the fatty acid component per kilogram of the milk yield after the first week after calving.

26. The method of claim 20, wherein the third level comprises at least 0.4 grams of the fatty acid component per kilogram of the body weight during the fresh cow period.

27. The method of claim 20, wherein the third level comprises at least 8 grams of the fatty acid component per kilogram of the milk yield during the peak lactation period.

28. The method of claim 20, wherein the third level comprises about 10 grams of the fatty acid component per kilogram of the milk yield during the peak lactation period.

29. The method of claim 20, wherein the third level comprises at least 8 grams of the fatty acid component per kilogram of the milk yield during the constant phase of lactation.

30. The method of claim 20, wherein the third level comprises about 10 grams of the fatty acid component per kilogram of the milk yield during the constant phase of lactation.

31. The method of claim 20, wherein the third level comprises at least 8 grams of the fatty acid component per kilogram of the milk yield during the late lactation period.

32. The method of claim 20, wherein the third level comprises about 10 grams of the fatty acid component per kilogram of the milk yield during the late lactation period.

33. The method of claim 1, wherein the milk yield is kilograms of milk produced per day.

34. The method of claim 1, wherein the at least one fatty acid component has a melting point of at least about 40° C.

35. The method of claim 1, wherein the at least one fatty acid component has a melting point of about 60° C.

36. The method of claim 1, wherein the at least one fatty acid component has a melting point of about 40° C. to about 80° C.

37. The method of claim 1, wherein the at least one saturated fatty acid component comprises about 70% to about 100% of the palmitic acid compound by weight.

38. The method of claim 1, wherein the at least one fatty acid component comprises stearic acid.

39. The method of claim 1, wherein the at least one fatty acid component comprises less than about 30% stearic acid by weight.

40. The method of claim 1, wherein the at least one fatty acid component comprises free palmitic acid and free stearic acid.

41. The method of claim 1, wherein the at least one fatty acid component comprises at least about 70% free palmitic acid by weight and less than about 30% free stearic acid by weight.

42. The method of claim 1, wherein the ruminant feed product is substantially free of trans-fatty acids.

43. The method of claim 1, wherein the ruminant feed product comprises about 0.5% to about 5% trans-fatty acids by weight.

44. The method of claim 1, wherein the palmitic acid compound comprises at least one of free palmitic acid and a palmitic acid derivative.

45. The method of claim 1, wherein the palmitic acid compound comprises at least one of the following: free palmitic acid, palmitate triglyceride, sodium palmitate, calcium palmitate, magnesium palmitate, ammonium palmitate, and derivatives thereof.

46. The method of claim 1, wherein the ruminant feed product comprises at least one nutrient component.

47. The method of claim 46, wherein the at least one nutrient component comprises at least one of the following: carnitine, at least one glucogenic precursor, at least one vitamin, at least one mineral, at least one amino acid, and at least one amino acid derivative.

48. The method of claim 46, wherein the at least one nutrient component comprises at least one of the following glucogenic precursors: glycerol, propylene glycol, molasses, propionate, glycerin, propane diol, and calcium propionate.

49. The method of claim 46, wherein the at least one nutrient component comprises at least one of the following vitamins: vitamin A, vitamin C, vitamin D, vitamin E, vitamin B1, vitamin B2, vitamin K, vitamin B₁, vitamin B₂, vitamin B₃, vitamin B₅, vitamin B₆, vitamin B₇, vitamin B₉, vitamin B₁₂, and vitamin Bₚ.

50. The method of claim 46, wherein the at least one nutrient component comprises at least one of the following minerals: calcium, sodium, magnesium, phosphorous, potassium, manganese, zinc, selenium, copper, iodine, iron, cobalt, and molybdenum.

51. The method of claim 46, wherein the at least one nutrient component comprises at least one of the following amino acids: histidine, alanine, isoleucine, arginine, leucine, asparagine, lysine, aspartic acid, methionine, cysteine, phenylalanine, glutamic acid, threonine, glutamine, tryptophan, glycine, valine, ornithine, proline, selenocysteine, serine, tyrosine, and derivatives thereof.

52. The method of claim 1, wherein the ruminant produces about 7 kilograms to about 10 kilograms of colostrum in a first milking after calving.

53. The method of claim 1, wherein feeding the ruminant according to the feeding program results in an increase in milk production by the ruminant relative to a substantially similar ruminant not fed according to the feeding program

54. The method of claim 1, wherein feeding the ruminant according to the feeding program results in an increase in fat content of milk produced by the ruminant relative to a substantially similar ruminant not fed according to the feeding program.

55. The method of claim 1, wherein feeding the ruminant according to the feeding program results in an increase in milk production of about 5% to about 10% by the ruminant relative to a substantially similar ruminant not fed according to the feeding program.

56. The method of claim 1, wherein feeding the ruminant according to the feeding program results in an increase in fat content of milk produced by the ruminant of about 10% relative to a substantially similar ruminant not fed according to the feeding program.

57. A method of feeding a ruminant, the method comprising:
determining a body weight of the ruminant and a milk yield of the ruminant; and
providing a ruminant feed product to the ruminant for ingestion according to a feed program;
wherein the feed product comprises at least one fatty acid component comprising at least about 70% of a palmitic acid compound by weight;
wherein the feed program comprises:
feeding the ruminant feed product to the ruminant at a first level of at most about 0.04 grams of the fatty acid component per kilogram of the body weight during a first time period, wherein the first time period comprises from about 6 week before calving to about 3 weeks before calving,
feeding the ruminant feed product to the ruminant at a second level of at least 0.04 grams of the fatty acid component per kilogram of the body weight during a second time period, wherein the second time period comprises a close-up dry cow period, and
feeding the ruminant feed product to the ruminant at a third level of at least 0.4 grams of fatty acid component per kilogram of the body weight during a third time period, wherein the third time period comprises at least one of a fresh cow period, a peak lactation period, a constant phase of lactation period, and a late lactation period.

58. The method of claim 57, wherein the third level comprises about 10 grams of the fatty acid component per kilogram of the milk yield during at least one of a fresh cow period, a peak lactation period, a constant phase of lactation period, and a late lactation period.

59. The method of claim 57, wherein the at least one fatty acid component has a melting point of at least about 40° C.

60. The method of claim 57, wherein the at least one fatty acid component has a melting point of about 60° C.

61. The method of claim 57, wherein the at least one fatty acid component has a melting point of about 40° C. to about 80° C.

62. The method of claim 57, wherein the at least one saturated fatty acid component comprises about 80% to about 90% of the palmitic acid compound by weight.

63. The method of claim 57, wherein the at least one fatty acid component comprises stearic acid.

64. The method of claim 57, wherein the at least one fatty acid component comprises less than about 30% stearic acid by weight.

65. The method of claim 57, wherein the at least one fatty acid component comprises free palmitic acid and free stearic acid.

66. The method of claim 57, wherein the at least one fatty acid component comprises at least 70% free palmitic acid by weight and less than about 30% free stearic acid by weight.

67. The method of claim 57, wherein the ruminant feed product is substantially free of trans-fatty acids.

68. The method of claim 57, wherein the ruminant feed product comprises about 0.5% to about 5% trans-fatty acids by weight.

69. The method of claim 57, wherein the palmitic acid compound comprises at least one of free palmitic acid and a palmitic acid derivative.

70. The method of claim 57, wherein the palmitic acid compound comprises at least one of the following: free palmitic acid, palmitate triglyceride, sodium palmitate, calcium palmitate, magnesium palmitate, ammonium palmitate, and derivatives thereof.

71. The method of claim 57, wherein the ruminant feed product comprises at least one nutrient component.

72. The method of claim 71, wherein the at least one nutrient component comprises at least one of the following: carnitine, at least one glucogenic precursor, at least one vitamin, at least one mineral, at least one amino acid, and at least one amino acid derivative.

73. The method of claim 71, wherein the at least one nutrient component comprises at least one of the following glucogenic precursors: glycerol, propylene glycol, molasses, propionate, glycerin, propane diol, and calcium propionate.

74. The method of claim 71, wherein the at least one nutrient component comprises at least one of the following vitamins: vitamin A, vitamin C, vitamin D, vitamin E, vitamin B1, vitamin B2, vitamin K, vitamin B₁, vitamin B₂, vitamin B₃, vitamin B₅, vitamin B₆, vitamin B₇, vitamin B₉, vitamin B₁₂, and vitamin Bₚ.

75. The method of claim 71, wherein the at least one nutrient component comprises at least one of the following minerals: calcium, sodium, magnesium, phosphorous, potassium, manganese, zinc, selenium, copper, iodine, iron, cobalt, and molybdenum.

76. The method of claim 71, wherein the at least one nutrient component comprises at least one of the following amino acids: histidine, alanine, isoleucine, arginine, leucine, asparagine, lysine, aspartic acid, methionine, cysteine, phenylalanine, glutamic acid, threonine, glutamine, tryptophan, glycine, valine, ornithine, proline, selenocysteine, serine, tyrosine, and derivatives thereof.

77. The method of claim 57, wherein the ruminant produces about 7 kilograms to about 10 kilograms of colostrum in a first milking after calving.

78. The method of claim 57, wherein feeding the ruminant according to the feeding program results in an increase in milk production by the ruminant relative to a substantially similar ruminant not fed according to the feeding program.

79. The method of claim 57, wherein feeding the ruminant according to the feeding program results in an increase in fat content of milk produced by the ruminant relative to a substantially similar ruminant not fed according to the feeding program.

80. The method of claim 57, wherein feeding the ruminant according to the feeding program results in an increase in milk production of about 5% to about 10% by the ruminant relative to a substantially similar ruminant not fed according to the feeding program.

81. The method of claim 57, wherein feeding the ruminant according to the feeding program results in an increase in fat content of milk produced by the ruminant of about 10% relative to a substantially similar ruminant not fed according to the feeding program.

82. A system for feeding a ruminant, the system comprising:
a feed source configured to provide a ruminant feed product for ingestion by the ruminant, the ruminant feed product comprising at least one fatty acid component comprising at least about 70% of a palmitic acid compound by weight;
at least one feeding element having access to the feed source, the at least one feeding element being configured to feed the ruminant feed product to the ruminant according to a feed program, the feed program comprising:
feeding the ruminant feed product to the ruminant at a first level during a first time period, wherein the first level does not exceed about 0.04 grams of the fatty acid component per kilogram of the body weight,
feeding the ruminant feed product to the ruminant at a second level during a second time period, wherein the second level comprises at least 0.04 grams of the fatty acid component per kilogram of the body weight, and
feeding the ruminant feed product to the ruminant at a third level during a third time period, wherein the third level comprises at least 0.4 grams of the fatty acid component per kilogram of the body weight.

83. The system of claim 82, further comprising at least one ruminant identification device configured to identify at least one ruminant and to adjust the feed program to correspond with the at least one ruminant.

84. The system of claim 82, wherein the first time period comprises a far-off dry cow period.

85. The system of claim 82, wherein the first time period comprises from about 6 week before calving to about 3 weeks before calving.

86. The system of claim 82, wherein the first level comprise a ruminant feed dry matter from about 1.5% to about 2.5% of the body weight.

87. The system of claim 82, wherein the first level is substantially free of saturated fatty acid component.

88. The system of claim 82, wherein the first level does not exceed 0.02 grams of the fatty acid component per kilogram of the body weight.

89. The system of claim 82, wherein the second time period comprises a close-up dry cow period.

90. The system of claim 82, wherein the second time period is about 3 weeks before calving to about calving.

91. The system of claim 82, wherein the second level comprises a first concentration during the third week before calving, a second concentration during the second week before calving, and a third concentration during the first week before calving.

92. The system of claim 91, wherein the first concentration comprises at least 0.04grams of the fatty acid component per kilogram of the body weight.

93. The system of claim 91, wherein the first concentration comprises from about 0.04 grams to about 0.08 grams of the fatty acid component per kilogram of the body weight.

94. The system of claim 91, wherein the first concentration comprises about 0.05 grams of the fatty acid component per kilogram of the body weight.

95. The system of claim 91, wherein the second concentration comprises at least 0.08 grams of the fatty acid component per kilogram of the body weight.

96. The system of claim 91, wherein the second concentration comprises from about 0.08 to about 0.16 grams of the fatty acid component per kilogram of the body weight.

97. The system of claim 91, wherein the second concentration comprises about 0.12 grams of the fatty acid component per kilogram of the body weight.

98. The system of claim 91, wherein the third concentration comprises at least 0.14 grams of the fatty acid component per kilogram of the body weight.

99. The system of claim 91, wherein the third concentration comprises from about 0.14 grams to about 0.22 grams of the fatty acid component per kilogram of the body weight.

100. The system of claim 91, wherein the third concentration comprises about 0.18 grams of the fatty acid component per kilogram of the body weight.

101. The system of claim 82, wherein the third time period is from about calving to a far-off dry cow period.

102. The system of claim 82, wherein the third time period comprises a fresh cow period, a peak lactation period, a constant phase of lactation period, and a late lactation period.

103. The system of claim 102, wherein the third level comprises a ruminant feed dry matter from about 4% to about 7% of the body weight

104. The system of claim 102, wherein the third level comprises from about 0.4 grams to about 0.8 g of the fatty acid component per kilogram of the body weight during the first week after calving.

105. The system of claim 102, wherein the third level comprises at most 0.5 grams of the fatty acid component per kilogram of the body weight during the first week after calving.

106. The system of claim 103, wherein the third level comprises about 10 grams of the fatty acid component per kilogram of the milk yield after the first week after calving.

107. The system of claim 102, wherein the third level comprises at least 0.4 grams of the fatty acid component per kilogram of the body weight during the fresh cow period.

108. The system of claim 102, wherein the third level comprises at least 8 grams of the fatty acid component per kilogram of the milk yield during the peak lactation period.

109. The system of claim 102, wherein the third level comprises about 10 grams of the fatty acid component per kilogram of the milk yield during the peak lactation period.

110. The system of claim 102, wherein the third level comprises at least 8 grams of the fatty acid component per kilogram of the milk yield during the constant phase of lactation.

111. The system of claim 102, wherein the third level comprises about 10 grams of the fatty acid component per kilogram of the milk yield during the constant phase of lactation.

112. The system of claim 102, wherein the third level comprises at least 8 grams of the fatty acid component per kilogram of the milk yield during the late lactation period.

113. The system of claim 102, wherein the third level comprises about 10 grams of the fatty acid component per kilogram of the milk yield during the late lactation period.

114. The system of claim 82, wherein the at least one fatty acid component has a melting point of about 40° C. to about 80° C.

115. The system of claim 82, wherein the ruminant feed product is substantially free of trans-fatty acids.

116. The system of claim 82, wherein the ruminant feed product comprises about 0.5% to about 5% trans-fatty acids by weight.

117. The system of claim 82, wherein the palmitic acid compound comprises at least one of free palmitic acid and a palmitic acid derivative.

118. The system of claim 82, wherein the palmitic acid compound comprises at least one of the following: free palmitic acid, palmitate triglyceride, sodium palmitate, calcium palmitate, magnesium palmitate, ammonium palmitate, and derivatives thereof.

119. The system of claim 82, wherein feeding the ruminant according to the feeding program results in an increase in milk production by the ruminant relative to a substantially similar ruminant not fed according to the feeding program

120. The system of claim 82, wherein feeding the ruminant according to the feeding program results in an increase in fat content of milk produced by the ruminant relative to a substantially similar ruminant not fed according to the feeding program.

121. The system of claim 82, wherein feeding the ruminant according to the feeding program results in an increase in milk production of about 5% to about 10% by the ruminant relative to a substantially similar ruminant not fed according to the feeding program.

122. The system of claim 82, wherein feeding the ruminant according to the feeding program results in an increase in fat content of milk produced by the ruminant of about 10% relative to a substantially similar ruminant not fed according to the feeding program.
